(19) United States
(12) Patent Application Publication
(10) Pub. No.: US 2017/0156786 A1
(43) Pub. Date: Jun. 8, 2017

(54) Radiowave currents electrode with adjacent active and inactive sections

(71) Applicant: Jon C. GARITO, Hewlett Harbor, NY (US)

(72) Inventor: Jon C. GARITO, Hewlett Harbor, NY (US)

(21) Appl. No.: 14/757,201

(22) Filed: Dec. 4, 2015

(51) Int. Cl. A61B 18/14

(52) U.S. Cl. CPC A61B 18/1402; A61B 2018/1412; A61B 2018/00077

(57) ABSTRACT

A radiowave current electrode having a shank for connection to a source of radiowave current or electrosurgical RF energy and at one end an active radiowave current electrode, the active end being shaped to perform a radiowave current procedure involving the application of the RF energy from an electrically conductive active surface of the active end to tissue to modify the tissue, the active surface having a plurality of spaced outwardly-projecting regions each shaped such that its outermost region is equal to or narrower than its innermost region, and such that more of the RF energy emanating from the active surface and passing to the tissue occurs via the outwardly-projecting regions. As a further feature, an adjacent section of the active end is coated with a substantially transparent insulating layer consisting essentially of a parylene plastic to render it inactive. As still a further feature, a thin layer of an RF excitable pigment is positioned below or mixed with the insulating layer such that when the pigment glows in response to the presence of RF on the electrode, the glow is visible to a practioner using the electrode.

This invention relates to novel radiowave current electrodes, and in particular to radiowave current electrodes for performing various surgical procedures including nail matrixectomy, nail spicule, and other podiatry and non-podiatry procedures requiring insertion into tissue of a radiowave current electrode with adjacent active and inactive sections to selectively modulate certain tissue regions while avoiding damage to adjacent healthy tissue regions.

### BACKGROUND OF THE INVENTION

Reference is made to U.S. Pat. No. 4,517,975 (herein the “‘975 patent”, of which I am one of the inventors, the contents of which are herein incorporated by reference, for a good description for understanding the present invention.

Radiowave current procedures for humans and animals are well established in the medical and dental arts. In the referenced ‘975 patent, the typical procedure (described therein as electrosurgical) involves generating a high frequency current, typically of the order of 0.5-4 MHz with a maximum output power of typically 30-150 Watts, and applying the resultant radio-frequency (RF) energy by way of an electrode to human or animal tissue. Different types of currents can be employed for different procedures. For example, fully rectified, fully filtered currents can be used for cutting tissue, fully rectified, non-filtered currents can be used for cutting with coagulation, partially rectified current can be used for hemostasis, and spark gap currents can be used for fulguration and dessication techniques. Such equipment, sometimes referred to as electrosurgical equipment, is available from many suppliers. Various electrodes configurations are also available; for example, metal needles for making incisions, wire loops, round or diamond shaped, for planing and contouring tissue, balls for coagulation and hemostasis, and scalpel shapes for incisions and excision of tissue. In many of these known electrode configurations, an electrically conductive shank, for mounting in the radiowave current handpiece, has a working end that is electrically conductive, usually metallic, and is fully exposed, so that all sides of the electrode working end are capable of transmitting the high frequency currents (herein referred to as “Radio-Frequency currents or RF energy”) to the tissue.

Humans and animals can suffer from a condition commonly known as ingrown nail (hypertrophy of the unguia labia or unguis incarnatus). The nail plate is rooted under a tissue fold at the digit proximal end and grows over a nail bed or matrix toward the distal end under lateral tissue folds in the so-called lateral grooves. The healthy nail should be rooted only at the proximal end. Ingrown nail results when the nail roots under the lateral folds. This results in laceration of the adjacent tissue, with possible pain, swelling and infection. The known surgical procedure, called matrixectomy, is to excise the unwanted or extraneous root. Merely removing the nail plate section adjacent the extraneous root will not prevent recurrence of the symptoms; the entire extraneous root must be excised and precautions taken to prevent re-rooting of the nail along the lateral grooves. The nail lateral edges or margin fit snugly into the groove and normally there is a little less than 1 mm of space between the nail margin and the nail lateral wall or lip. In the RF surgical procedure described in the above-referenced ‘975 patent, a radiowave current or electrosurgical electrode with a specially designed tip whose working end is partly bare and partly insulated is used to selectively direct the RF energy by way of the bare part to only part of the tissue with which the electrode tip is in contact or is adjacent. In the preferred form as described in that patent, the electrode tip is spade shaped, one flat side of which is bare metal and the opposite flat side of which is coated with an electrical insulator. When such an electrode tip is contacted to tissue, the RF energy is supposed to exit only via the bare electrode side. Tissue facing or contacting the coated side remains unexposed and unaffected by the RF energy.

Experience has indicated that in some situations, the exiting RF currents are not always confined to the flat bare side of the electrode. One problem is that the RF energy sometimes flows to the edges of the electrode (known as the “edge effect”) rather than to the middle or the total area of the exposed metal flat side. As a result there is poorer contact with the diseased tissue and the practitioner must spend more time attempting to ablate the matrix cells. This overaggressive application can cause more heat to be introduced into the cells. The result may be inconsistent matrix cell thermal ablation causing delayed nail regrowth, pain and delayed healing.

Another problem that often occurs with the existing design of the matrixectomy electrode is that the Teflon coating used on the insulated side of the spade end wears quickly due to heat. Moreover, it has been shown to have low biocompatability, low dialectric strength, and a low resistance to gamma sterilization, which can introduce problems into its use with the electrosurgical electrode.

### BRIEF SUMMARY OF THE INVENTION

An object of the invention is an improved electrode for carrying out radiowave current or electrosurgical procedures for modulating tissue.

Another object of the invention is an improved electrode for carrying out radiowave current procedures using RF energy in which the electrode comprises active regions or sections for selectively modulating tissue with the RF energy adjacent to inactive regions or sections for protecting tissue against the RF energy.

Another object of the invention is an improved surgical procedure for the removal of symptomatic spicule growth in the nail groove of a patient.

Still another object of the invention is an improved electrode for the radiowave current treatment of ingrown nail.

In accordance with a feature of the invention, a more efficient radiowave current electrode is obtained by including a series of furrows or corrugations or depressions or points forming plural spaced outwardly projecting regions narrower, which is preferred, or of the same size at the end than at their base in the exposed metal side of a spade-like or oval-like or flat active end of the electrode. Preferably these furrows or corrugations or depressions form spaced sharpened regions such as a point (like a sharkfin). The presence of these pointed spaced projecting regions causes the RF energy to be distributed in a more even way via these points across the active side of the electrode.

The pointed furrows on the electrode spade end would also serve another important purpose. As part of the standard treatment protocol for matrixectomy, it is necessary to remove excess granulation tissue from the site. Various elevator type instruments are used for this purpose. The pointed furrows on the electrode of the invention make unnecessary these elevators as the electrode of the invention is an excellent tool to remove the granulation tissue and manually smooth the nail bed surface.

In accordance with another feature of the invention, the insulating coating covering the non-active side of the electrode, typically Teflon, is replaced by an insulating plastic material known as Parylene available from Specialty Coating Systems, Inc. of Indianapolis, Ind. 46278, as well as Berwind Corporation as an SCS Micro Resist Antimicrobial Parylene Technology material, which is especially characterized for this application by excellent bio compatability, dielectric strength, excellent wear and adhesion properties, excellent resistance to gamma, and excellent chemical and moisture barrier properties.

In accordance with another feature of the invention, the inactive or protected side of the electrode is coated with an RF activatable fluorescent pigment such that when the handpiece holding the electrode is activated to supply to the electrode RF energy, the RF will cause the pigment to glow and can be visually seen by the practitioner through the thin skin fold under the cuticle while carrying out a matrixectomy procedure. This avoids another problem with the existing matrixectomy electrode in that when placed under the cuticle skin fold and activated the doctor often can't tell if the electrode is receiving RF energy and actually working as intended.

In accordance with another feature of the invention, a rounded electrode with a smooth surface used in a nail spicule removal procedure as described in another of my patents U.S. Pat. No. 5,683,386, whose contents are also incorporated herein by reference, is enhanced by the addition around its working end of corrugations or furrows or depressions terminating in reduced diameter spaced ends, for example, pointed ends. This improvement offers the same advantages of more uniformly spreading the RF energy where tissue ablation is desired in a more consistent manner.

### DESCRIPTION OF THE DRAWINGS

The invention will now be described in greater detail with respect to several exemplary embodiments, taken in conjunction with the annexed drawings wherein:

FIGS. 1-6 are perspective (from 2 different positions), side, plan, cross-section and partial perspective views, respectively, of one form of radiowave current electrode in accordance with the invention, especially useful in matrixectomy;

FIGS. 7-9 are partly schematic views illustrating use of the electrode of FIG. 1 in a nail surgical procedure;

FIGS. 10-13 are perspective, end and side views, respectively, of another form of radiowave current electrode in accordance with another feature of the invention for various medical procedures;

FIGS. 14-17 are perspective, end and side views, respectively, of another form of radiowave current electrode in accordance with another feature of the invention for various medical procedures;

FIGS. 18, 19 and 21 are perspective, cross-sectional and side views, respectively, of another form of RF energy electrode in accordance with another feature of the invention for treating various medical problems.

FIG. 20 is a perspective view of a variant of the electrode show in FIG. 18;

FIGS. 22 and 23 illustrate use of the electrode of FIG. 15 in a nail matrixectomy procedure.

FIGS. 24-26 are perspective and side views, respectively, of another form of radiowave current electrode in accordance with another feature of the invention for treating various medical problems.

FIGS. 27-33 are views from different perspectives of another form of electrode in accordance with another feature of the invention for treating various medical problems.

### DETAILED DESCRIPTION OF PREFERRED EMBODIMENTS

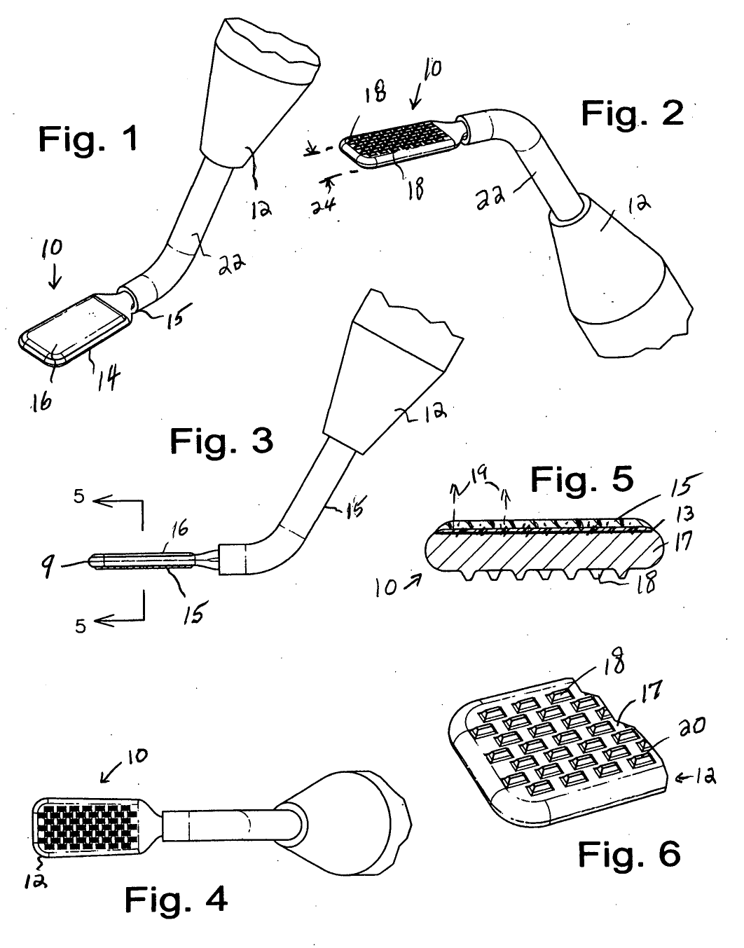

FIG. 1 is a perspective view of one form of the electrode 10 according to the invention mounted in a conventional radiowave current or electrosurgical handpiece of which only the front end 12 of the handpiece is shown. The view of the electrode is similar to that shown in the ‘975 patent. In this embodiment, the electrode 10 is spade-shaped, the inactive bottom surface 14 is coated with an insulating layer 15 (not shown here), and the active surface 16, shown schematically but in more detail in FIG. 2 (which shows the electrode 10 in the reversed position but also with the active surface uppermost) is covered with a plurality of spaced outwardly projecting regions 18 whose outermost dimension is preferably smaller than its innermost dimension. In this embodiment, each individual region 18 (see FIG. 6 as an example) is a polygon with slanted sides such that each outermost region is essentially a line 20 of reduced length. In this embodiment, the spaced outwardly projecting regions 18 form a series of equally spaced parallel lines. The electrode shank 22 is electrically-conductive but covered with an electrically-insulating coating 15, and the base 17 and projections 18 of the active end are also electrically-conductive. The opposite inactive side as well as the shank is coated with an insulating layer 15 so that only the exposed side 16 with the projections 18 are electrically active (see the cross-section of FIG. 5). As explained above, when the handpiece 12 is activated by the apparatus to which it is plugged into, the RF energy passes down the electrode shank to the bare active side with the projections which tend to concentrate and spread evenly the RF energy across the face of the active side so that it mainly emanates from the small area lines 20 of the projections. There are enough projections present (24 in this example) so that essentially the entire active side is electrically active, thus ensuring that the RF energy can be focused by the practitioner wherever the active side of the electrode is placed in contact with tissue.

As one example only, which is not to be considered limiting, the overall length of the electrode is approximately 2-3 inches long, specifically 2 ⅜ inches in the preferred example shown, with a shank diameter of 1/16- 3/32 inches. The spade tip shown is approximately 11 mm long, which can widen from 1/16 inches wide at its proximal end to 4 mm wide at its distal end (in the FIG. 1 embodiment, the front and rear widths of the base are alike). The uncoated tip thickness 9 is approximately 0.01 in. thick, and the insulating coating 16 has a thickness of about 0.004 in. Other shapes and thicknesses are also suitable. For matrixectomy, a suitable range of metal thickness is from about 0.006-0.050 in., and for the coating from 0.002-0.020 in. It is preferred to provide a family of four electrodes from the largest as above described down to the smallest having a maximum spade width of 1 mm, in 1 mm steps, with corresponding spade lengths of 5-11 mm. This is illustrated in FIG. 2 with the width indicated by numeral 24. FIGS. 1 and 2 show essentially the same electrode with active side upright but in reversed positions for the convenience of the practioner.

FIGS. 4 and 6 show a similar active end of the electrode but in which the outwardly projecting regions 18 have again been sub-divided into individual regions 18 but still aligned in parallel lines. The parallel line arrangement is not essential. The projections can be arranged in circular arrays, or in concentric circles, or even randomly spaced across the face of the active electrode side so long as enough projections are present to distribute the RF energy generally evenly across the face of the active side.

The manufacture of the electrode can be conventional using standard stamping, machining and similar techniques and may be constructed of malleable metal, typically brass or stainless steel, so that the practitioner may bend it into a desired configuration. Specific techniques for forming the metallic parts are described in detail in the referenced patents as is also the preparation and application of the plastic insulating layer to the desired areas of the active end. Another way of producing the inventive structure is by conductively gluing or tightly adhering in a known way various metal fragments or particles with sharp surfaces onto the active bare electrode surface. Among the ways available are machining into the bare electrode surface, and embossing or appliquing onto the flat metal spade or oval shaped metal. Metal particles such as silver, gold, or even tungsten can be attached to the flat metal spatula or oval shaped working end by electroplating or brazing or both. The metal fragments are preferably made of a higher conductivity metal such as silver or gold so they are more likely to convey the RF energy than the lower conductivity base metal. The metal particles may typically be 5 to 100 microns in size.

FIG. 5 also illustrates another feature of the invention that can be used with the embodiments described herein as well as with prior art embodiments that also use a working end that has active and inactive adjacent sections with the inactive sections coated with a plastic layer to prevent RF energy from modulating tissue that it may be in contact with during the procedure. In procedures such as matrixectomy, where the working end is inserted under tissue, sometimes it is difficult for the practioner to know whether the electrode is actually active with RF and where it is difficult to actually see the effects under the tissue. In accordance with this feature of the invention, a thin layer of an RF actuable pigment is placed under the insulating layer. The insulating layer is very thin, typically, 0.004 inches, and is virtually transparent. Therefore, if a thin layer of the pigment is placed underneath, and when it glows when RF is applied to the electrode, the glow will be visible to the practitioner through the covering plastic layer and the thin layer of skin tissue and then he or she can be assured that the active side is applying RF energy to the tissue areas desired. This is illustrated in FIG. 5 with the thin plastic designated 15, and the thin pigment layer underneath designated 13 and with several broken arrows 19 indicating the glow from the pigment when the electrode is activated with RF. The thin plastic layer covers and protects the pigment from being eroded as the working end is inserted and removed from under the skin fold. F fluorescent powders, commercially available, are suitable as the pigment for this purpose. It is also possible to blend the fluorescent powders with the plastic material, whether of the prior art or parylene type, so that the two are integrated into one layer. It is also possible to use shrink tubing protective covers, also mixed with the pigment, to serve as the electrically-insulating RF-responsive coating for the non-active sections.

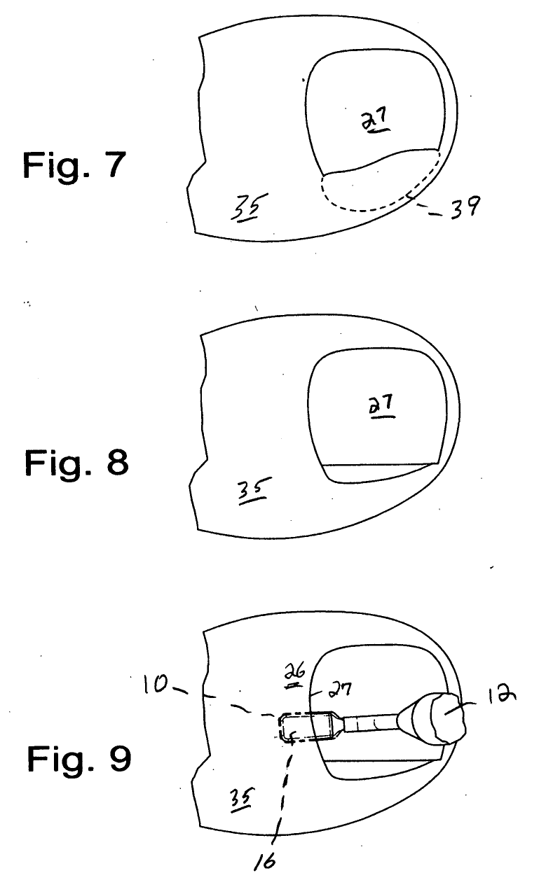

FIGS. 7-9 illustrate use of the electrode of the invention on a human or animal digit 35 in a matrixectomy procedure. The spade tip 10 is inserted under the tissue fold 26 covering the nail plate 27 edge until the bare bottom active side 16 is located over the tissue section to be destroyed, designated in dashed lines by 39. When the radiowave current or electrosurgical equipment is energized, after appropriate grounding of the patient, eg., to patient's calf, the high frequency currents or RF energy flows through the bare outwardly projecting regions 18 of the tip 10 and into the adjacent tissue 39. The operating conditions are chosen so as to destroy the cells adjacent the exposed active electrode side 16. As one example, which is not to be considered limiting, power is applied for 1-2 seconds while moving the electrode. Then, the equipment is deenergized for 10-15 seconds, and power reapplied for 1-2 seconds if necessary and the procedure repeated until the extraneous root cells are destroyed. Using conventional equipment available from known manufacturing sources, it is recommended to use either the partially filtered or the fulguration current at about a 30 watt power level. Other currents and powers may be best with equipment from other suppliers, which is readily determined by simple experimentation. The tissue portions 26 abutting the upper coated side 14 of the spade tip 12 are unaffected by the high frequency currents.

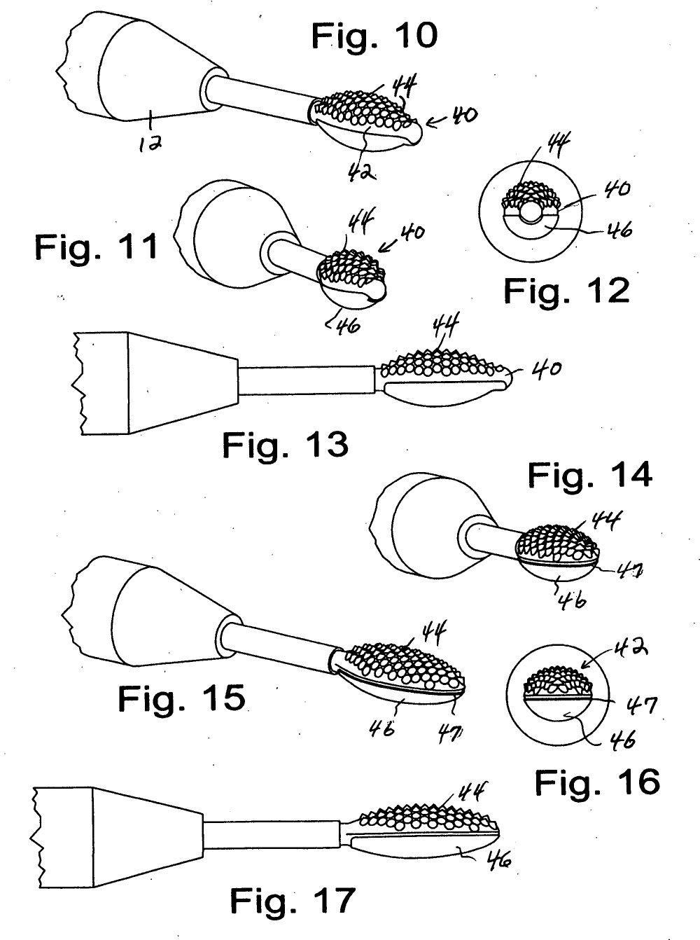

FIGS. 10-17 illustrate shapes other than the spade shape of FIG. 1 of the active end that would also be suitable to treat various ingrown nail situations. In these cases, the electrode tips 40 are generally oval shaped with the top side 42 as shown provided with the plurality of spaced outwardly projecting regions 44 which preferably have smaller dimensions at the outermost tip, and preferably are generally pointed (see FIG. 17). As before, the top sides 42 shown are of bare metal and the bottom inactive side is made insulating with a plastic layer 46. The actual shape of the projecting regions 44 is not critical so long as they tend to narrow outwardly forming preferred paths for the RF energy and therefore distributing the RF more evenly across the surface than if as in the prior art the surface were smooth or flat. In the embodiments of FIGS. 14-17, the darker lines 47 at the oval boundaries indicate extension of the plastic layer 46 on the underside over the edges of the oval to prevent or limit the edge effect, i.e., emanation of RF energy from the edges.

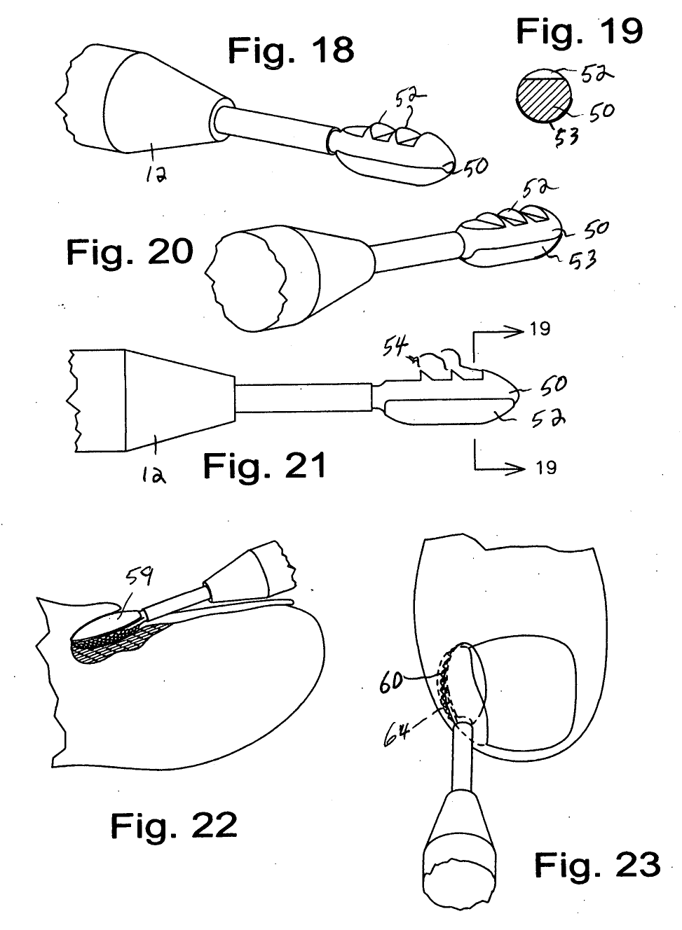

FIGS. 18-21 show still another suitable configuration to distribute the RF energy more uniformly over the active surface of the electrode tip. In this case, the normally smooth surface of the active end 50 is formed with three upstanding edges 52 again coming to a pointed end 54 as illustrated in FIG. 21. A plastic layer 53 covers the bottom inactive surface. This construction is simpler but in certain cases may not be as effective as the surface with the more numerous projections illustrated in the previous figures.

FIGS. 22-23 show still another suitable configuration to distribute the RF energy more uniformly over the active surface of the electrode tip 59 again in an oval shape. In this case, the plural outstanding spaced projections 60 are concentrated along one edge 64 of the tip of the electrode. The shapes of the projections are more similar to those in the embodiments of FIGS. 10-13 but in this embodiment are concentrated just along the edges of the active tip where the practioner may need to concentrate the RF energy under certain circumstances. But the underlying concept is similar, namely, to provide plural outstanding narrowing projections in place of the normally smooth surface of the prior art active end to distribute the RF energy more uniformly.

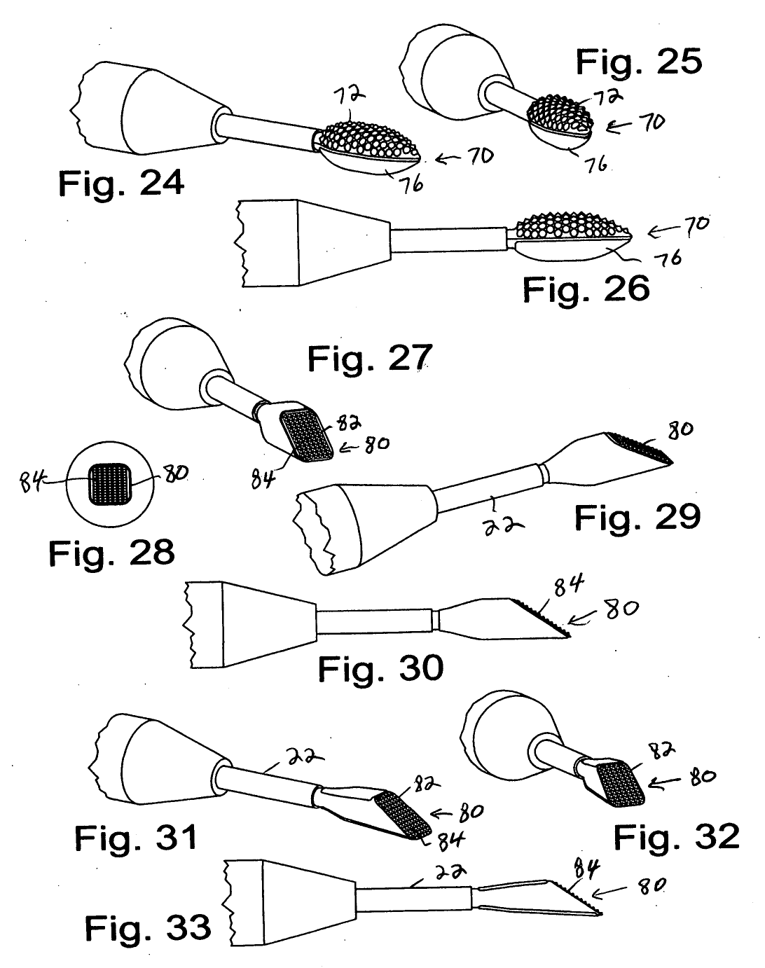

FIGS. 24-33 show other configurations of an oval electrode tip according to the invention. In FIGS. 24-26, the overall shape of the electrode tip 70 is somewhat less elongated than in the embodiments of FIG. 15 but the plural outwardly projecting projections 72 from the active side 74 are about the same shape, and as before the bottom inactive side is coated with an electrically-insulating plastic layer 76.

FIGS. 27-33 show other variations of the shape of the active tip 80 of the electrode according to the invention. In these cases, the overall shape is chisel-shaped, flat but angled to the shank 22 axis. The flat active side 82 is covered with the plurality of spaced outwardly extending projections 84 whose narrowed ends spread the RF energy more evenly across the active side of the electrode. This embodiment also illustrates the variation wherein the outwardly projections 84 are formed by conductively gluing or tightly adhering in a known way various metal fragments with sharp surfaces onto the active bare electrode surface 82. The metal fragments 84 are given sharp edges or points and are preferably made of a higher conductivity metal such as silver or gold so they are more likely to convey the RF energy than the lower conductivity base metal. The different shapes of these tips according to the invention are useful in nail spicule and related procedures. It will also be noted that the embodiments of FIGS. 27-33 have many more individual projections 84 than for example the embodiment of FIG. 24, and the size of the projections is also smaller than those in the other embodiments. This demonstrates that it is the replacement of the flat surface with the bumpy surface formed by the projections that characterizes the embodiments of the invention and provides the improved performance.

Some typical examples are, for example, for the tip of FIG. 26 a length of 11 mm. For the shapes of FIGS. 27-33, which can be described as chisel shaped, the length can be 8-10 mm with the bumpy part adding another 3 mm. The protrusion or bump 82 can be, for example 0.435 inches long and come to a point of 0.078 inches. Again, these dimensions are preferred but are not critical. For the chisel-shaped ends, with sharper pointed protrusions, some typical dimensions for the pointed ends are a height of about 0.003 inches for the points with a point spacing of about 0.006 inches.

The entire electrode preferably comprises an elongated one-piece metal body having at one end the extended shank 22 and at the opposite end, the working end or tip, with opposed sides. The uncoated tip thickness 9 (perpendicular to the plane of the drawing) (FIG. 3) is approximately 0.01 in. thick, and the coating 16 has a thickness of about 0.004 in. Other shapes and thicknesses are also suitable. For matrixectomy, a suitable range of metal thickness is from about 0.006-0.050 in., and for the coating from 0.002-0.020 in. It is preferred to provide a family of four electrodes from the largest as above described down to the smallest having a maximum spade width of 1 mm, in 1 mm steps, with corresponding spade lengths of 5-11 mm. The latter is illustrated in FIGS. 2, with the width indicated by numeral 24. The overall tip dimensions recited in the two referenced patents are also suitable for the electrode of the present invention. The partly insulated, partly bare electrode tip of the invention with its numerous protrusions in the same or other configurations should also prove useful in other electrosurgical procedures for medical, dental or veternarian uses where the electrode tip is in contact with different tissues or tissue parts only some of which are to be selectively destroyed by currents from the bare part of the electrode tip while other contacted tissues or parts are to be preserved by the protective coating on the coated part of the electrode tip.

While the invention has been described in connection with specific embodiments thereof, those skilled in the art will recognize that various modifications are possible within the principles enunciated herein and thus the present invention is not to be limited to the specific embodiments disclosed.

What is claimed is:

1. A radiowave current electrode for delivering RF energy and having at one end an active radiowave current electrode, the active end being shaped to perform a radiowave current procedure involving the application of the RF energy from an electrically conductive active surface of the active end to tissue to modify the tissue, the active surface having a plurality of spaced outwardly-projecting regions each shaped such that its outermost region is substantially equal to or narrower than its innermost region, and such that more of the RF energy emanating from the active surface and passing to the tissue occurs via the plural outwardly-projecting regions.

2. A radiowave current electrode as claimed in claim 1, said working tip having adjacent active and inactive sections, the plural outwardly-projecting regions covering the active section, an electrically-insulating layer of plastic covering the inactive section to prevent RF energy from modulating tissue adjacent the inactive section.

3. A radiowave current electrode as claimed in claim 2, wherein the active end has a spade shape or oval shape or chisel shape, and the active section is bare and covered with the projecting regions.

4. A radiowave current electrode as claimed in claim 3, wherein the each of the projecting regions come to a generally pointed end.

5. A radiowave current electrode as claimed in claim 2, wherein the plastic is parylene.

6. A radiowave current electrode as claimed in claim 2, further comprising a layer of an RF responsive florescent pigment positioned under or mixed with the electrically-insulating coating such that, in response to RF energy applied to the active end, the pigment will glow such that it can be visually seen by a practitioner to indicate the presence of RF energy at the active end.

7. A radiowave current electrode as claimed in claim 1, wherein the active end is spade-shaped, the active surface is electrically-conductive, and the non-active surfaces are non-electrically-conductive, and the procedure is matrixectomy.

8. A radiowave current electrode as claimed in claim 1, wherein the active end is tapered or oval-shaped, the active surface is electrically-conductive, and the procedure is nail spicule removal.

9. A radiowave current electrode as claimed in claim 1, wherein the active end is tapered or oval-shaped or flat, the active surface is electrically-conductive, and the projecting regions are formed by high conductivity sharp particles conductively adhered to the active surface.

10. A radiowave current electrode for delivering radiowave current RF energy and having at one end an active radiowave current electrode, the active end having active and inactive adjacent sections with the active section being shaped to perform a radiowave current procedure involving the application of the RF energy from an electrically conductive active surface of the active section to tissue to modify the tissue, further comprising an electrically-insulating layer covering the inactive section to prevent RF energy from modulating tissue when in contact with the inactive section, the electrically-insulating layer consisting essentially of a parylene plastic material.

11. A radiowave current electrode as claimed in claim 10, wherein the active end has a spade shape or oval shape or chisel shape, and the active section is bare and covered with spaced projecting regions to more uniformly distribute the RF energy to the tissue.

12. A radiowave current electrode for delivering electrosurgical RF energy and having at one end an active radiowave current electrode, the active end having active and inactive adjacent sections with the active section being shaped to perform a radiowave current procedure involving the application of the RF energy from an electrically conductive active surface of the active section to tissue to modify the tissue, further comprising a substantially transparent electrically-insulating layer covering the inactive section to prevent RF energy from modulating tissue when in contact with the inactive section, further comprising a thin layer of an RF responsive florescent pigment positioned under or mixed with the electrically-insulating layer such that, in response to RF energy applied to the active end, the pigment will glow such that it can be visually seen through the transparent electrically-insulating layer by a practitioner to indicate the presence of RF energy at the active end.

13. A radiowave current electrode as claimed in claim 12, wherein the electrically-insulating layer consists essentially of a parylene plastic material.

14. A radiowave current electrode as claimed in claim 12, wherein the active end has a spade shape or oval shape or chisel shape, and the active section is bare and covered with spaced outwardly projecting regions to more uniformly distribute the RF energy to the tissue.
